(19) United States
(12) Patent Application Publication
(10) Pub. No.: US 2026/0070125 A1
(43) Pub. Date: Mar. 12, 2026

(54) SYNTHESIS AND FUNCTIONALIZATION OF PARTICLES IN A BOTTOM-UP REACTION

(71) Applicant: Iowa State University Research Foundation, Inc., Ames, IA (US)

(72) Inventors: Connor Phelps Thorpe, Ames, IA (US); Shan Jiang, Ames, IA (US)

(21) Appl. No.: 19/323,561

(22) Filed: Sep. 9, 2025

Related U.S. Application Data
(60) Provisional application No. 63/692,265, filed on Sep. 9, 2024.

(51) Int. Cl. B22F 9/20; A61K 48/00; B22F 1/05; B22F 1/054; B22F 1/102

(52) U.S. Cl. CPC B22F 9/20; A61K 48/0033; B22F 1/05; B22F 1/054; B22F 1/056; B22F 1/102; B22F 2301/255; B22F 2304/056; B22F 2304/058; B22F 2304/10; B22F 2998/10; B22F 2999/00

(57) ABSTRACT

The synthesis of several functionalized particles without the need of additional delivery agents are made possible with unique bottom-up reactions. For example, the synthesis of gold nanoparticles from 300 nm-6000 nm in diameter are optimized size for use in biolistic delivery. A catalytic reaction in a simple aqueous phase can be carried out at room temperature (between 20° C. and 24° C.). A catalyst enables production of spherical particles of desirable size and narrow size distribution. Surface functionalization assists delivery of biomolecules (DNA, protein, RNA) without the need of additional delivery agent. The gold particles system improves the consistency of the particle sizes, controls the size distribution, increases the loading capacity, and improves the number of cells transfected, thereby making the biolistic process more efficient and consistent.

### CROSS REFERENCE TO RELATED APPLICATIONS

This application claims priority under 35 U.S.C. § 119(e) to provisional patent application U.S. Ser. No. 63/692,265, filed Sep. 9, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to biolistic particle delivery systems and related methods. More particularly, but not exclusively, the present disclosure relates to synthesis and functionalization of gold particles for ballistic delivery.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Biolistic delivery (often times called gene gun) stands as a versatile tool in genetic engineering, showing several advantages over its primary alternative, Agrobacterium-mediated delivery. Biolistic delivery is not species genotype or explant dependent and can deliver proteins. One important component of the biolistic delivery system is the “bullet”, which is commonly made from Au particles. Currently the commercial Au particles are often made through ballmilling, which produces a rough surface with heterogenous size distribution.

The ideal Au particle size for biolistic delivery ranges from three hundred nanometers to one and a half microns (300 nm-1.5 μm). However, synthesizing Au particles within this size range using solution-based reactions is challenging. Typical commercial particles are produced via top-down approaches, such as physical grinding or ball milling, resulting in heterogeneous particles with a large size distribution. Additionally, these commercially available particles are usually sold without any surface functionalization, making them difficult to suspend and leading to inconsistent results.

The most common gold nanoparticle synthesis method is the Turkevich Method developed in 1951, which utilizes a gold hydrochlorate solution reduced by sodium citrate at one hundred degrees Celsius (100° C.) and subsequently capped by citrate. The particle sizes obtained using this method range from sixteen nanometers to one hundred forty seven nanometers (16 nm-147 nm). The Brust Method is another common gold synthesis method achieving particle sizes from one and a half nanometers to two and one fifth nanometers (1.5-2.2 nm) using a two-phase reaction. Another more commonly used method these days is the seed-mediated growth method, which involves synthesizing seed gold particles, having a diameter between seven nanometers and ten nanometers (7 nm-10 nm), at one hundred degrees Celsius (100° C.) and subsequently growing them generation after generation until they reach particles as large as two hundred nanometers (200 nm) which are spherical and monodisperse before the particles started to aggregate and crashed out of the solution. Lastly, the digestive ripening method is another method of preparing gold nanoparticles at high temperatures from one hundred ten to one hundred forty degrees Celsius (110° C.-140° C.), which obtains particles anywhere from four to twenty nanometers (4 nm-20 nm) depending on the temperature. All of these methods require complex synthesis, high temperatures, sometimes organic solvent and tedious steps, and cannot make particles larger than two hundred nanometers (200 nm), which are required for efficient biolistic delivery into cells.

Thus, there exists a need in the art for a new gold particle synthesis that allows for precise control of particle sizes and mono-dispersity with a smooth surface.

### SUMMARY

A new gold particle synthesis that uses a novel reducing agent and results in one hundred percent (100%) yield of gold particles at room temperature in about thirty minutes (30 min). A new application of surface functionalized gold particles in biolistic delivery requires no binding agent and salt for complexing with DNA, RNA, and proteins. This simplifies the process and loads DNA much more efficiently. The invention further decreases the time, reagents, and steps required to bind DNA to gold particles and is more user-friendly.

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to control the size and morphology of the gold particles. Changing the size and morphology can be accomplished by changing a concentration or a volume of the reducing agent and a capping agent used in the reaction.

It is still yet a further object, feature, and/or advantage of the present disclosure to optimize stirring of the reaction. The stirring rate is preferably between three hundred and two thousand revolutions per minute (300 rpm-2000 rpm) but is typically done at one thousand revolutions per minute (1000 rpm). The reaction time is typically three hours (3 hrs) but some particles are synthesized in less than thirty minutes (30 minutes). The longer the reaction time, the more particles you yield up until around three hours (3 hrs) when it caps out.

It is still yet a further object, feature, and/or advantage of the present disclosure to employ a reducing agent with certain reducing properties. For example, 4-aminophenol and o-phenylenediamine work well because they lead to large uniform particle sizes with high yield and can be synthesized in less than 3 hours. Sodium hydroxide, sodium borohydride, ascorbic acid, hydroquinone, and sodium citrate, do not work well.

The synthesis and functionalization of gold particles for biolistic delivery can be accomplished under a wide variety of conditions. For example, a temperature of the bottom-up synthesis reaction is between zero degrees Celsius (0° C.) and one hundred degrees Celsius (100° C.).

The synthesis and functionalization of gold particles for biolistic delivery disclosed herein can be used in a wide variety of applications. For example, both polyvinylpyrrolidone (PVP)-capped gold particles and cysteamine-capped particles can be used for biolistic delivery. PVP-capped particles are negatively charged and require a binding agent like the typical commercial gold particles. The cysteamine-capped particles are positively charged and do not require a binding agent. It is also worth noting that Au particles can be used for applications beyond biolistic delivery, such as contrast agent for computed tomography (CT) scans and as drug delivery vehicles.

Methods can be practiced which facilitate use, manufacture, assembly, maintenance, and repair of biolistic particle delivery systems which accomplish some or all of the previously stated objectives.

According to some aspects of the present disclosure, a bottom-up synthesis reaction to form gold particles having a diameter between three hundred nanometers and six microns (300 nm-6 μm), more preferably between five hundred nanometers and four microns (500 nm and 4 μm), even more preferably between one micron and three microns (1 μm-3 μm), and most preferably about two microns (2 μm) comprises a capping agent; a reducing agent; and a gold precursor. A temperature of the bottom-up synthesis reaction is between zero degrees Celsius (0° C.) and one hundred degrees Celsius (100° C.).

According to some additional aspects of the present disclosure, the bottom-up synthesis reaction of claim 1, wherein the gold precursor is gold (III) chloride hydrate (HAuCl4). Alternatively, the gold precursor can be selected from the group consisting of: Chloroauric Acid (HAuCl₄); Gold(I) Chloride (AuCl); Gold(I) Thiosulfate (Au(S₂O₃)₂³⁻); Gold(III) Nitrate (Au(NO₃)₃); Gold(III) Acetate (Au(C₂H₃O₂)₃); Gold(I) Thioglucose (Au(TG)); Gold(I) Tetrafluoroborate (AuBF₄); Gold(III) Sulfate (Au₂(SO₄)₃).

According to some additional aspects of the present disclosure, the capping agent comprises polyvinylpyrrolidone (PVP), hydroxyethyl cellulose (HEC), polyvinyl alcohol (PVA), and polyethylene glycol (PEG).

According to some additional aspects of the present disclosure, the reducing agent comprises 4-aminophenol or o-phenylenediamine.

According to some additional aspects of the present disclosure, the temperature of the reaction is a room temperature between 20° C. and 24° C.

According to some additional aspects of the present disclosure, the capping agent comprises cysteamine and the synthesis reaction is free from a binding agent and salt for complexing with DNA.

According to some additional aspects of the present disclosure, the yield of the bottom-up synthesis reaction is approximately one hundred percent.

According to some other aspects of the present disclosure, a method comprises using a synthesis reaction to form gold particles having a diameter between three hundred nanometers and two microns (300 nm-2 μm), wherein a temperature of the reaction is between zero degrees Celsius (0° C.) and one hundred degrees Celsius (100° C.); and surface functionalizing the gold particles with a binding agent.

According to some additional aspects of the present disclosure, the binding agent comprises highly positively charged molecules.

According to some additional aspects of the present disclosure, the method further comprises capping the gold particles with cysteamine.

According to some additional aspects of the present disclosure, the gold particles are monodisperse gold particles.

According to some additional aspects of the present disclosure, the method further comprises stirring the reaction at a rate between three hundred and two thousand revolutions per minute (300 rpm-2000 rpm).

According to some additional aspects of the present disclosure, the gold particles are synthesized in less than thirty minutes.

According to some additional aspects of the present disclosure, the synthesis reaction is a bottom-up synthesis reaction.

According to some additional aspects of the present disclosure, the method further comprises controlling a size and a morphology of the gold particles through selecting a concentration or a volume of a reducing agent and a capping agent used in the synthesis reaction.

According to some other aspects of the present disclosure, a biolistic delivery system for delivery of the gold particles synthesized using any one or more of the methods described in any one or more of the preceding paragraphs.

According to some additional aspects of the present disclosure, the gold particles are used for causing a (i) transient transformation, (ii) a stable transformation, and/or (iii) gene editing with (a) DNA, (b) RNA, and/or (c) a protein.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows gold nanoparticle SEM images (left: 300 nanometers; right: 1.6 microns) prepared by the new synthesis method.

FIG. 2 shows SEM images of synthesized gold particles of different sizes between one hundred fifty nanometers and sixteen hundred nanometers (150 nm-1600 nm) (upper-left: 150 nm-300 nm; upper-right: 400 nm-600 nm; lower-left: 700 nm-1000 nm; lower-right: 1400 nm-1600 nm).

FIG. 3 shows SEM images of synthesized gold particles with different morphologies (left: spherical particles; middle: Au rose particles; right: Au nanourchins particles).

FIG. 4 shows SEM images of synthesized gold particles with different surface functionalization (left: 1000 nm Au PVP; right: 1600 n, Au Cysteamine).

FIG. 5 shows SEM images of commercial gold particles and particles produced of various sizes.

FIG. 6 shows optical fluorescent images of GFP-DNA transfected onion epidermis cells with various commercial gold sources and gold particles produced using Spermidine and TransIT-2020 binding agents.

FIG. 7 shows optical fluorescent images of GFP-DNA transfected onion epidermis cells with gold particles produced modified with cysteamine (no binding agent needed).

FIG. 8 shows onion epidermis transient transfection performance of various commercial gold particle sources and gold produced.

FIGS. 9A-J show a morphology phase diagram and aspects associated therewith.

FIG. 9A shows mapped out the synthesis conditions that lead to different gold particle morphologies by varying the amount of PVP (capping agent) and 4-aminophenol (reducing agent) in the reaction while keeping the gold precursor concentration constant.

FIG. 9B shows SEM images of morphologies observed.

FIG. 9C shows an SEM image of a sphere monodisperse morphology.

FIG. 9D shows an SEM image of a cluster monodisperse morphology.

FIG. 9E shows an SEM image of a cauliflower monodisperse morphology.

FIG. 9F shows an SEM image of a raspberry monodisperse morphology.

FIG. 9G shows an SEM image of a urchin monodisperse morphology.

FIG. 9H captures an SEM image of the kinetics of the reaction, showing formation of polydisperse seed particles.

FIG. 9I captures an SEM image of the kinetics of the reaction, showing formation of irregular aggregates.

FIG. 9J captures an SEM image of the kinetics of the reaction, showing self-assembly of monodisperse supraparticles with different morphologies.

FIGS. 10A-10F evidence very clearly that reducing power in the synthesis depends less on the functional groups, but more on the benzene ring, allowing efficient lending of electrons for reduction.

FIG. 10A shows that reducing agents work due to the O-Phenylenediamine structure.

FIG. 10B shows that reducing agents work due to the 4-Aminophenol structure.

FIG. 10C shows that reducing agents work due to the Hydroquinone structure.

FIG. 10D shows that reducing agents work due to the O-Phenylenediamine structure.

FIG. 10E and FIG. 10F show reducing agents do not work because 4-Aminocyclohexanol and 2-Amino-1,3-Propanediol do not have a benzene ring, hence the reducing agents are weaker reducing agents and do not form nanoparticles.

FIGS. 11A-11F show characterization of commercial vendor gold particles.

FIG. 11A shows SEM images of the commercial particles being very polydisperse due to being ball-milled instead of synthesized for ASI 300 nm, InBio 400 nm, ASI 600 nm, BR 600 nm, ASI 1000 nm, and InBio 1600 nm.

FIG. 11B quantifies binding release in μg of the commercial particles being very polydisperse due to being ball-milled instead of synthesized for both the bound average and release average with respect to each of ASI 300 nm, InBio 400 nm, ASI 600 nm, BR 600 nm, ASI 1000 nm, and InBio 1600 nm.

FIG. 11C quantifies particle diameter in nm of the commercial particles being very polydisperse due to being ball-milled instead of synthesized for each of ASI 300 nm, InBio 400 nm, ASI 600 nm, BR 600 nm, ASI 1000 nm, and InBio 1600 nm.

FIG. 11D quantifies cell viability in % of the commercial particles being very polydisperse due to being ball-milled instead of synthesized for each of ASI 300 nm, InBio 400 nm, ASI 600 nm, BR 600 nm, ASI 1000 nm, and InBio 1600 nm; thereby supporting that this leads to larger and inconsistent aggregates being delivered into plant tissues, reducing the efficiency of delivery.

FIG. 11E quantifies drying diameter in nm of the commercial particles being very polydisperse due to being ball-milled instead of synthesized for each of ASI 300 nm, InBio 400 nm, ASI 600 nm, BR 600 nm, ASI 1000 nm, and InBio 1600 nm; thereby supporting that this leads to larger and inconsistent aggregates being delivered into plant tissues, reducing the efficiency of delivery.

FIG. 11F quantifies transfected cells of the commercial particles being very polydisperse due to being ball-milled instead of synthesized for each of ASI 300 nm, InBio 400 nm, ASI 600 nm, BR 600 nm, ASI 1000 nm, and InBio 1600 nm; thereby supporting that this leads to larger and inconsistent aggregates being delivered into plant tissues, reducing the efficiency of delivery.

FIGS. 12A-12H characterize synthesized spherical gold particles.

FIG. 12A captures SEM images showing size control over the particles as well as superior monodispersity for 400 nm, 600 nm, 1000 nm, and 1600 nm-gold particle size.

FIG. 12B quantifies particle diameter in nm for each of 400 nm, 600 nm, 1000 nm, and 1600 nm-gold particle size, thereby showing size control over the particles as well as superior monodispersity.

FIG. 12C quantifies statistical correlation among gold particle size (400 nm, 600 nm, 1000 nm, and 1600 nm) and mono-dispersity (in mL).

FIG. 12D quantifies statistical correlation among gold yield (in %) and mono-dispersity (in mL).

FIG. 12E shows binding and release efficiency (in μg) increases with smaller particles (higher surface area).

FIG. 12F shows cell viability (in %) increases with smaller particles.

FIG. 12G shows drying diameter (in nm) decreases with smaller particles.

FIG. 12H shows transfected cells decreases with smaller particles.

FIGS. 13A-F characterize different synthesized gold morphologies.

FIG. 13A quantifies intensity with respect to 2 Theta.

FIG. 13B shows average diameter (nm) with respect to each anisotropic structure. Urchin morphology particles lead to cells with the highest average diameter. Cauliflower morphology had the second highest average diameter.

FIG. 13C shows binding and release efficiency (in μg) increases with more anisotropic structures (more surface area).

FIG. 13D shows cell viability (in %) with respect to each anisotropic structure. Cauliflower morphology particles lead to the highest percentage of cell viability. Cluster morphology was the second best for cell viability.

FIG. 13E shows average diameter (nm) with respect to each anisotropic structure. Urchin morphology particles lead to cells with the highest average drying diameter. Cauliflower morphology had the second highest average drying diameter.

FIG. 13F shows transfected cells with respect to each anisotropic structure. Cauliflower morphology particles lead to the best performance, transfecting approximately ˜1575 cells on average. Spherical morphology was the second best at around ˜1450 cells.

FIGS. 14A-14F show functionalization of synthesis spherical, cauliflower, and urchin morphologies.

FIG. 14A diagrams a ligand exchange with PVP to replace with cysteamine to allow direct binding of DNA to the gold surface. There is no longer a need for an external delivery agent.

FIG. 14B evidences surface charge shows functionalization works. Cysteamine particles are very positively charged.

FIG. 14C shows rougher morphologies have higher average drying diameter (in nm).

FIG. 14D shows rougher morphologies have better binding and release. The functionalized particles bind and release slightly better than the plain particles with an external delivery agent.

FIG. 14E shows cell viability (in %) with respect to each of spherical, cauliflower, and urchin morphologies. Cauliflower morphology particles lead to the highest percentage of cell viability. Spherical morphology was the second best for cell viability.

FIG. 14F evidences functionalized cauliflower gold particles had the best performance with approximately ˜1750 cells transfected on average. Functionalized spherical particles were the second best with around ˜1500 cells on average.

FIGS. 15A-15D show that substituting the gold precursor with either a silver or copper precursor will still allow for a successful reaction.

FIG. 15A captures an SEM image showing silver particles formed with silver nitrate as the precursor.

FIG. 15B captures an SEM image showing copper particles formed with copper(II) nitrate trihydrate as the precursor.

FIG. 15C captures a zoomed-in SEM image showing silver particles formed with silver nitrate as the precursor.

FIG. 15D captures a zoomed-in SEM image showing copper particles formed with copper(II) nitrate trihydrate as the precursor.

FIG. 16 shows a biolistic particle delivery system.

FIGS. 17A-17B shows a microcarrier launch assembly usable with the biolistic particle delivery system of FIG. 16. FIG. 17A shows an assembled view. FIG. 17B shows a disassembled view.

FIG. 18 shows an elevation view of a target shelf usable with the biolistic particle delivery system of FIG. 16.

FIG. 19 shows the biolistic bombardment process using the biolistic particle delivery system of FIG. 16.

FIG. 20A shows a side elevation view components and controls on a helium driven gene gun.

FIG. 20B shows a side, partial cutaway view of a battery compartment of the helium driven gene gun of FIG. 20A.

FIG. 21A shows an exploded view of major components used for sample delivery with the helium driven gene gun of FIG. 21A.

FIG. 21B shows a cross-sectional view of a helium driven gene gun, emphasizing view of a high velocity stream of helium that accelerates gold particles coated with plasmids or RNA to velocities sufficient to penetrate and transform cells, both in vitro and in vivo.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

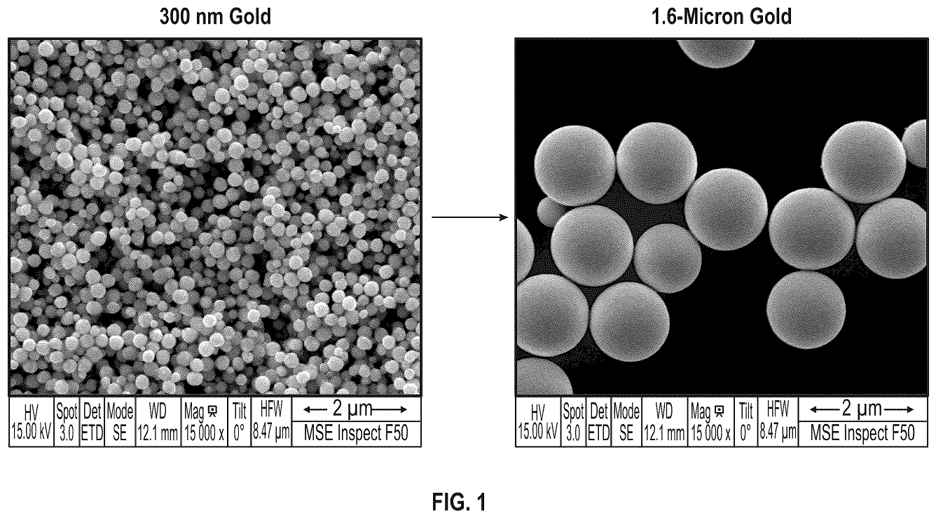

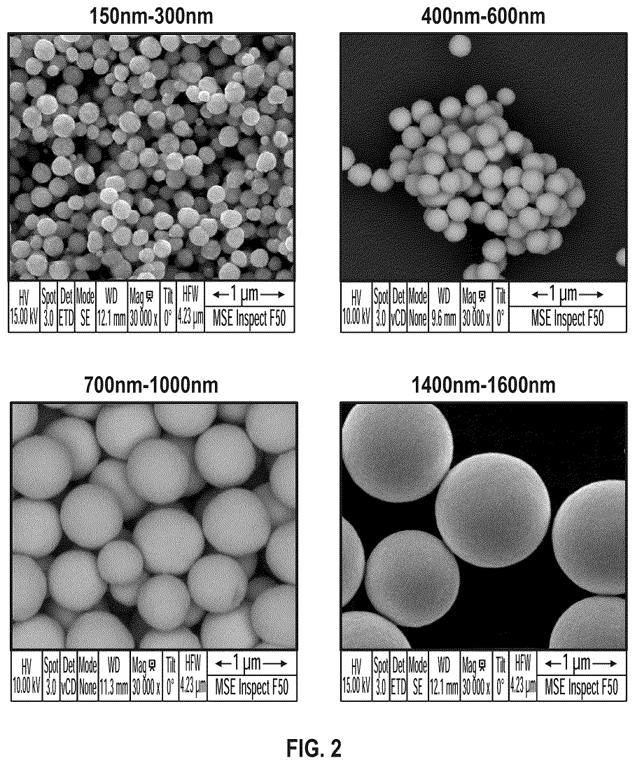

The present disclosure concerns a method to synthesize gold particles from three hundred nanometers to two microns (300 nm-6 μm) in size at room temperature (between 20° C. and 24° C.) using gold precursor, a stabilizing agent, and a novel reducing agent for gold in an aqueous solution, as shown in FIG. 1. Most importantly, the new method can obtain monodisperse spherical gold particles from three hundred nanometers to two microns (300 nm-2 μm) in diameter. The large monodisperse gold particles are achieved using a bottom-up synthesis method, as shown in FIG. 2.

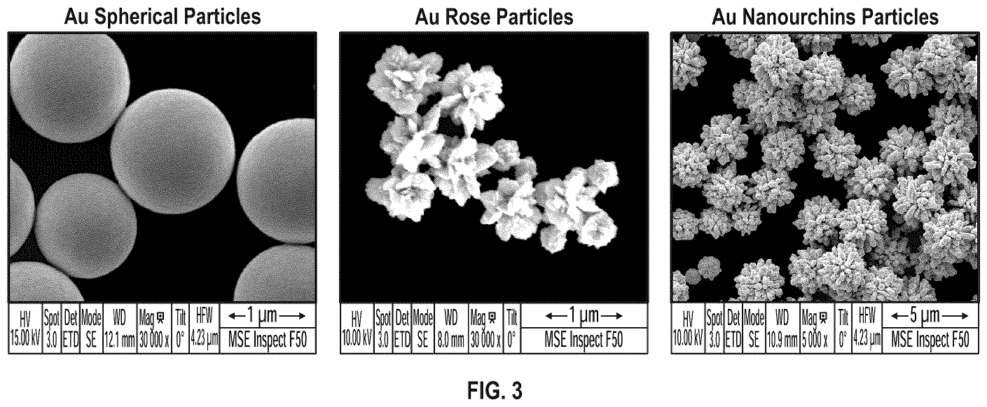

As an example, the gold synthesis can involve using gold (III) chloride hydrate (HAuCl4) as a precursor, PVP as a stabilizer, and 4-aminophenol as a reducing agent, with a stirring speed of eight hundred revolutions per minute (800 rpm) at room temperature (between 20° C. and 24° C.), resulting in monodisperse spherical particles in only thirty minutes to three hundred sixty (30 min-3600 min), as shown in FIG. 3. Many other reducing agents have been tested, including ascorbic acid and hydroquinone, but ascorbic acid and hydroquinone did not yield good results.

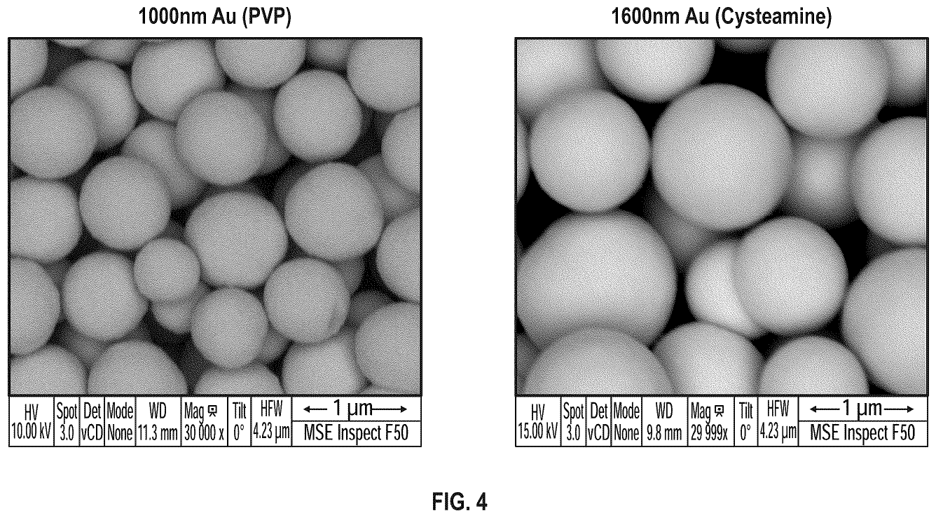

After the gold particles are synthesized and washed, a ligand exchange is performed at room temperature (between 20° C. and 24° C.) with cysteamine dissolved in water and mixed with the particles, replacing the PVP on the gold surface and effectively functionalizing the particles with a highly positively charged cysteamine molecule, as shown in FIG. 4. These cysteamine-functionalized gold particles of sizes three hundred nanometers to two microns (300 nm-2 μm) can then be used to bind and deliver highly negatively charged DNA molecules via biolistic delivery very efficiently. No cysteamine-functionalized gold particles were capable of being used to deliver DNA using biolistic delivery prior to the discoveries of the present disclosure.

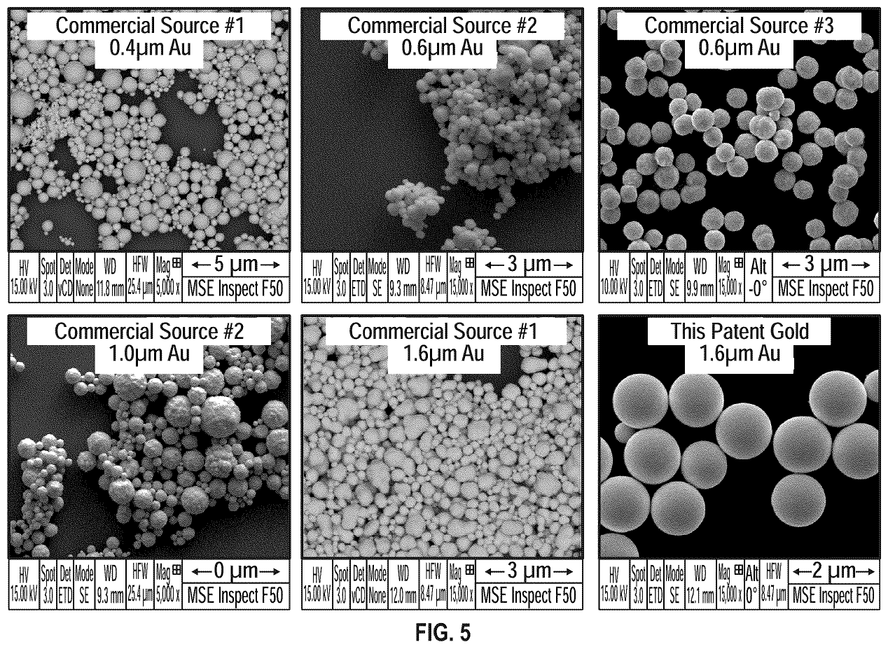

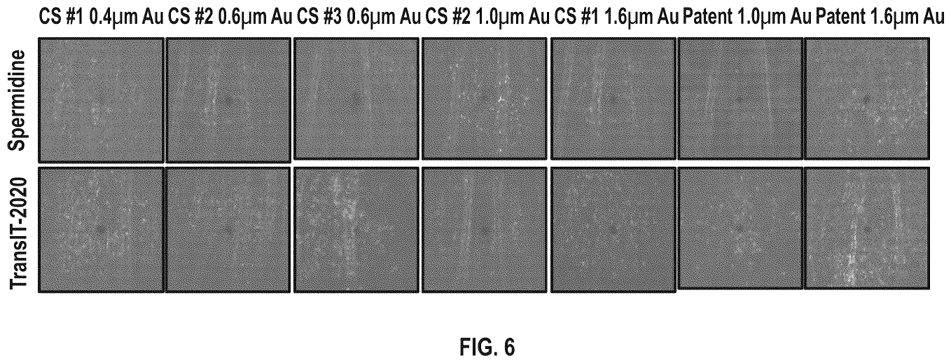

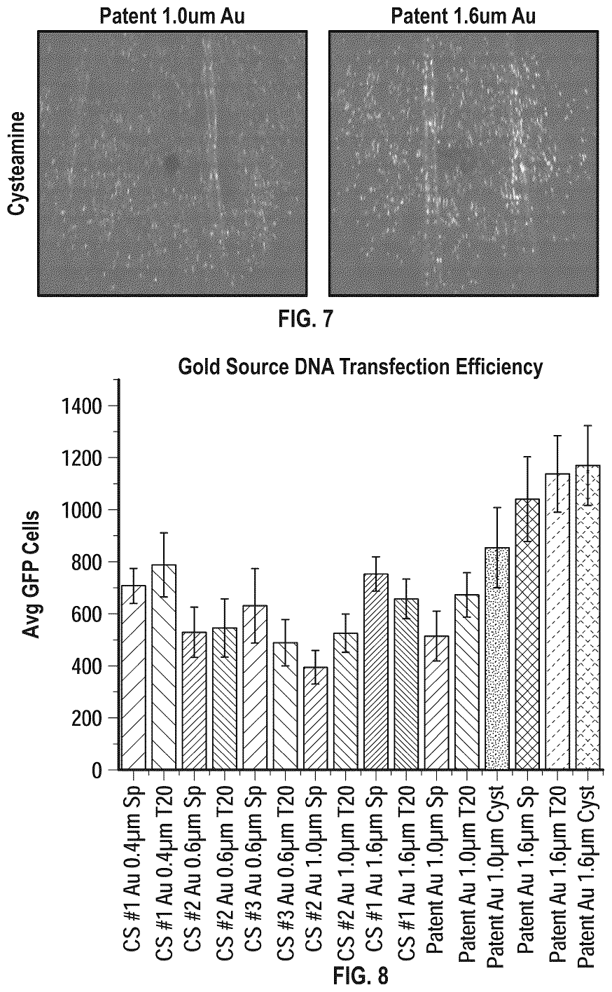

These cysteamine-functionalized gold particles of sizes 300 nm-6 μm in size can then be used to bind and deliver highly negatively charged DNA molecules via biolistic delivery very efficiently, as shown in FIG. 5. It is believed no cysteamine-functionalized gold particles have been used to deliver DNA, protein, or RNA using biolistic delivery. Moreover, the PVP-capped gold particles are negatively charged and can be used as a direct replacement for commercial gold particles with the use of a typical binding agent such as spermidine or TransIT-2020, as evidenced by FIG. 6 (optical fluorescent images of GFP-DNA transfected onion epidermis cells with various commercial gold sources and gold particles produced using Spermidine and TransIT-2020 binding agents). FIG. 7 evidences no binding agent is needed to GFP-DNA transfect onion epidermis cells with gold particles produced modified with cysteamine. FIG. 8 evaluates onion epidermis transient transfection performance of various commercial gold particle sources and gold produced.

Genetic engineering in plants is a large field with enormous potential. However, the fundamental tools used by both academia and industry for delivering genetic material to plant genomes are relatively limited, due to the need to circumvent the thick cell wall present in plant cells. While some are able to remove the cell wall to provide easier access, or hijack particular bacteria that have evolved this function, the most general method for doing so is to punch through the barrier using the momentum of a small, dense, DNA- or protein-coated metal particle.

Biolistic particle delivery is a method of transformation that uses helium pressure to introduce DNA-coated microcarriers into cells. Particle delivery transforms intact cells in culture. Microprojectile bombardment can transform such diverse targets as bacterial, fungal, insect, plant, and animal cells and intracellular organelles. Minimal pre- or post-bombardment manipulation is required. Biolistic particle delivery is regarded as being much easier and faster to perform than the tedious task of micro-injection.

This biological-ballistic (biolistic) method has been adopted around the world by university researchers as well as virtually all companies developing genetically modified crops. The main limiting factor when using the biolistic method is low transformation efficiencies. This is due to the small number of cells receiving the DNA/protein upon bombardment and the need to balance the destructive impact with effective cell penetration.

It is thus to be appreciated that the synthesized gold particles can be used in biolistic particle delivery systems and related methods.

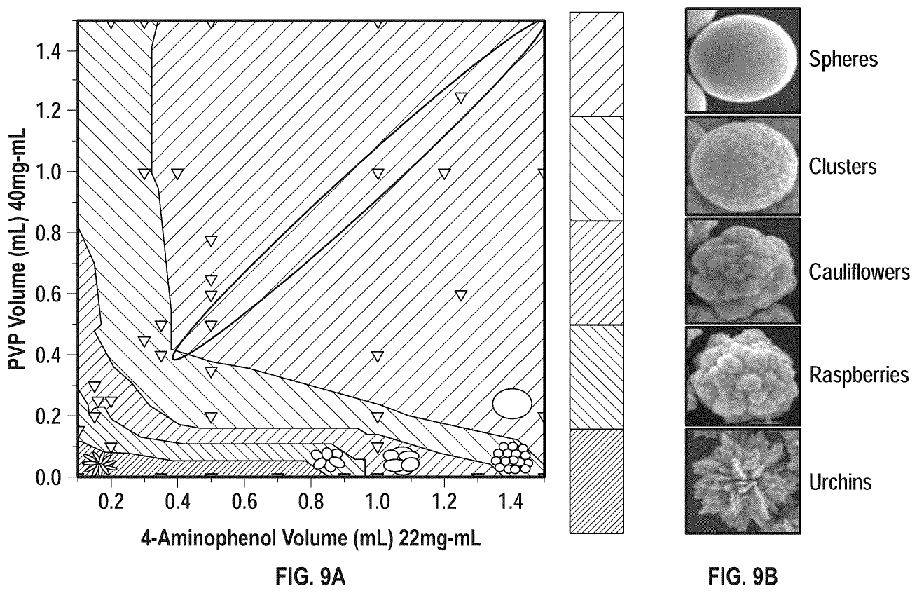

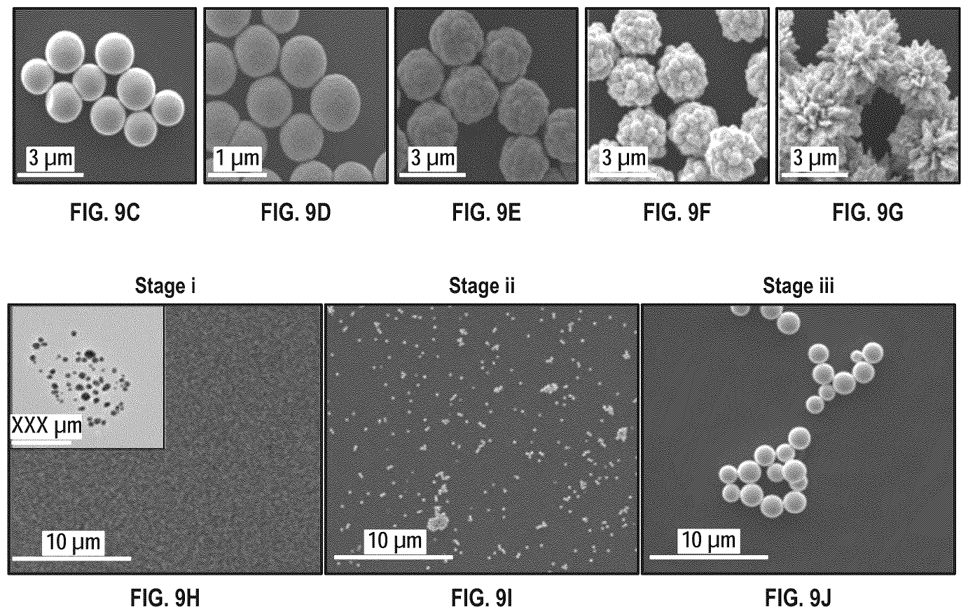

FIG. 9A maps out the synthesis conditions that lead to different gold particle morphologies by varying the amount of PVP (capping agent) and 4-aminophenol (reducing agent) in the reaction while keeping the gold precursor concentration constant. Viewing the graph such that the y-axis begins at 0.0 and runs from south (bottom) to north (top) and such that the x-axis begins at 0.0 and runs west (left) to east (right): the areas shaded with the loosest south-west to north-east lines correspond with spheres, an SEM image of the sphere morphology being captured at the very top (first row) of FIG. 9B; the area shaded with the loosest north-west to south-east lines correspond with clusters, an SEM image of the cluster morphology being captured at the middle-top (second row) of FIG. 9B; the areas shaded with the middle south-west to north-east lines correspond with cauliflowers, an SEM image of the cauliflower morphology being captured at the very top (third row) of FIG. 9B; the area shaded with the middle north-west to south-east lines correspond with raspberries, an SEM image of the raspberry morphology being captured at the middle-bottom (fourth row) of FIG. 9B; and the areas shaded with the densest south-west to north-east lines correspond with urchins, an SEM image of the urchin morphology being captured at the very bottom (fifth row) of FIG. 9B. FIGS. 9C-9G show SEM images of monodisperse morphologies observed with respect to spheres, clusters, cauliflowers, raspberries, and urchins, respectively. FIGS. 9H-9J evidence SEM images showing the kinetics of the reaction. First, formation of polydisperse seed particles, then formation of irregular aggregates, and finally self-assembly of monodisperse supraparticles with different morphologies.

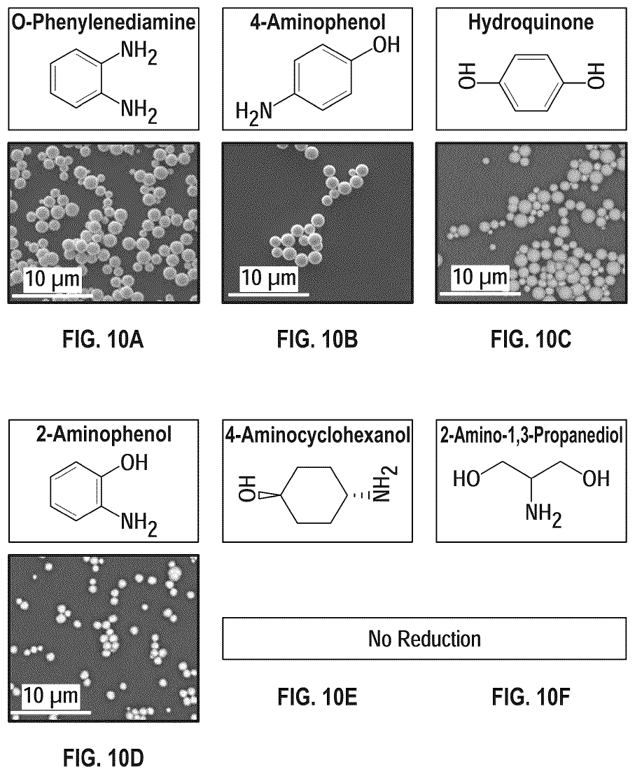

FIGS. 10A-10F evidence very clearly that reducing power in the synthesis depends less on the functional groups, but more on the benzene ring, allowing efficient lending of electrons for reduction. FIGS. 10A-10D show that reducing agents work due to the structure. FIGS. 10E-10F reducing agents do not work because they do not have a benzene ring, hence they are weaker reducing agents and do not form nanoparticles.

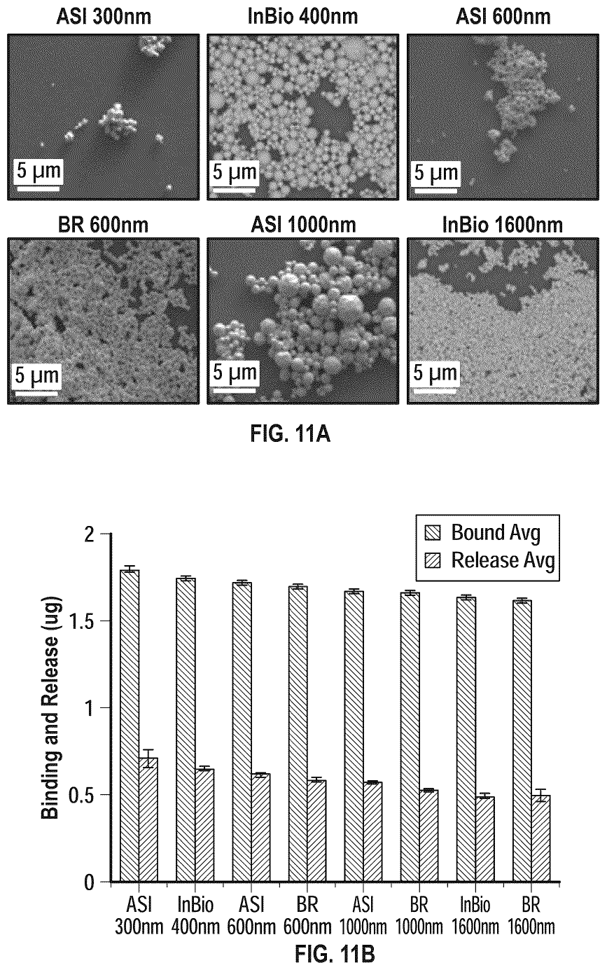

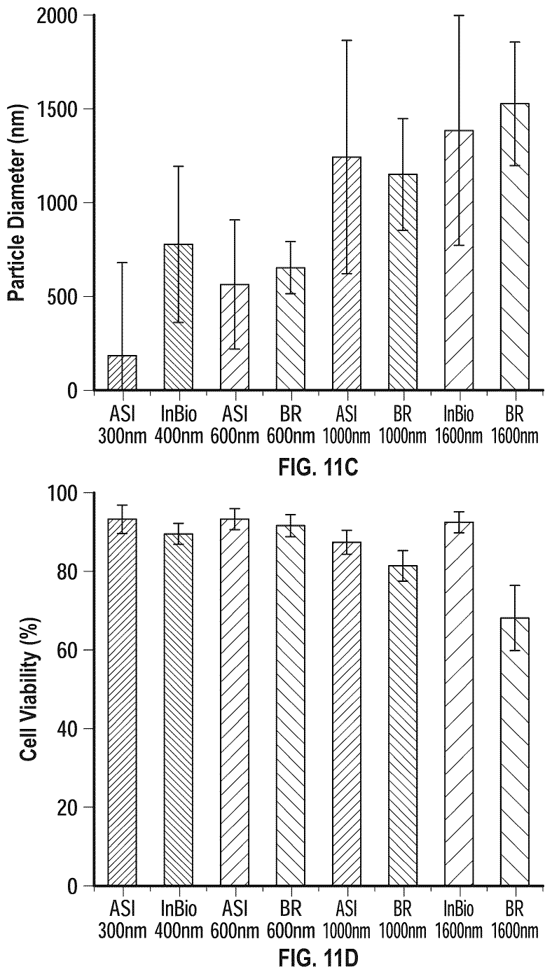

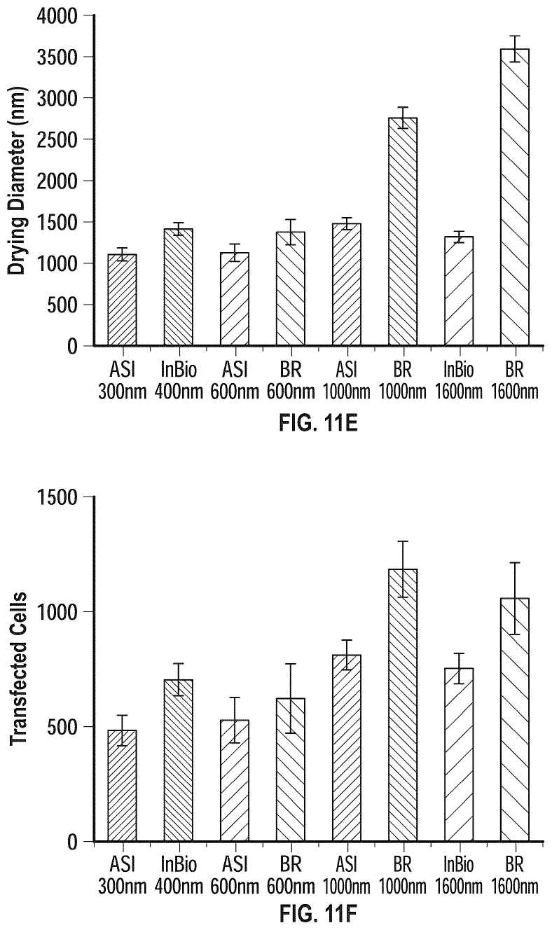

FIGS. 11A-11F show characterization of commercial vendor gold particles. FIGS. 11A-11C show the commercial particles are very polydisperse due to being ball-milled instead of synthesized. FIGS. 11D-11F show that this leads to larger and inconsistent aggregates being delivered into plant tissues, reducing the efficiency of delivery. The best performer was the Bio-Rad 1000 nm particles with ˜1150 cells transfected on average.

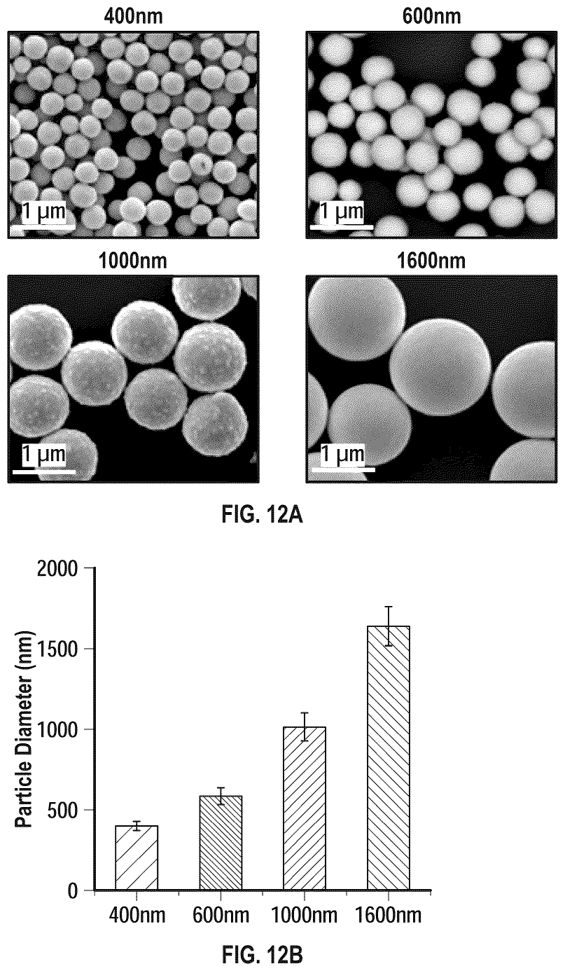

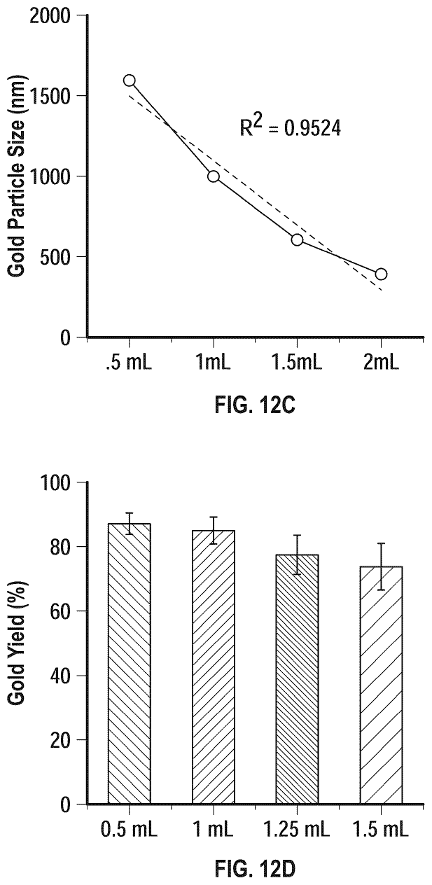

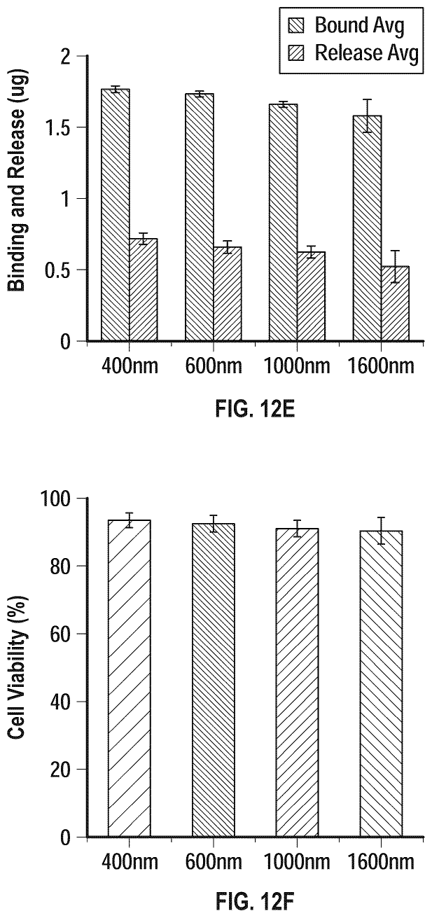

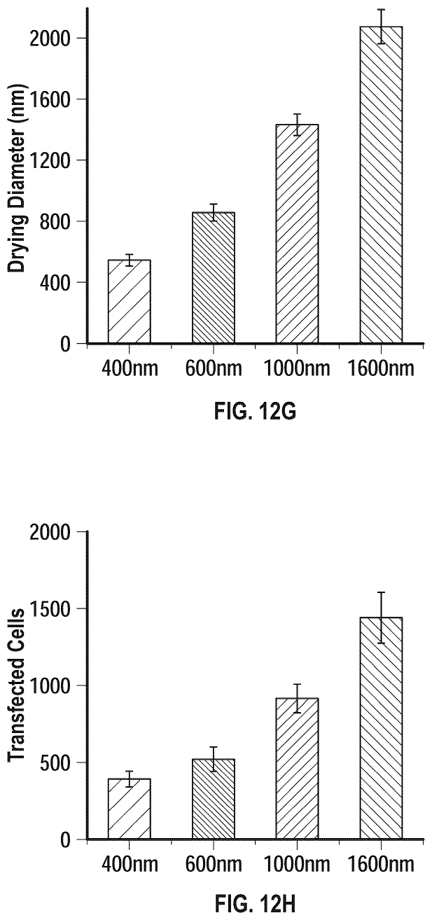

FIGS. 12A-12H show characterization of synthesized spherical gold particles. FIGS. 12A-12B show size control over the particles as well as superior monodispersity. FIG. 12E shows binding and release efficiency increases with smaller particles (higher surface area). FIGS. 12F-12H show that this leads to more uniform and smaller aggregates, resulting in more efficient delivery and less cell damage. The best result was with the 1600 nm particles with ˜1450 cells transfected.

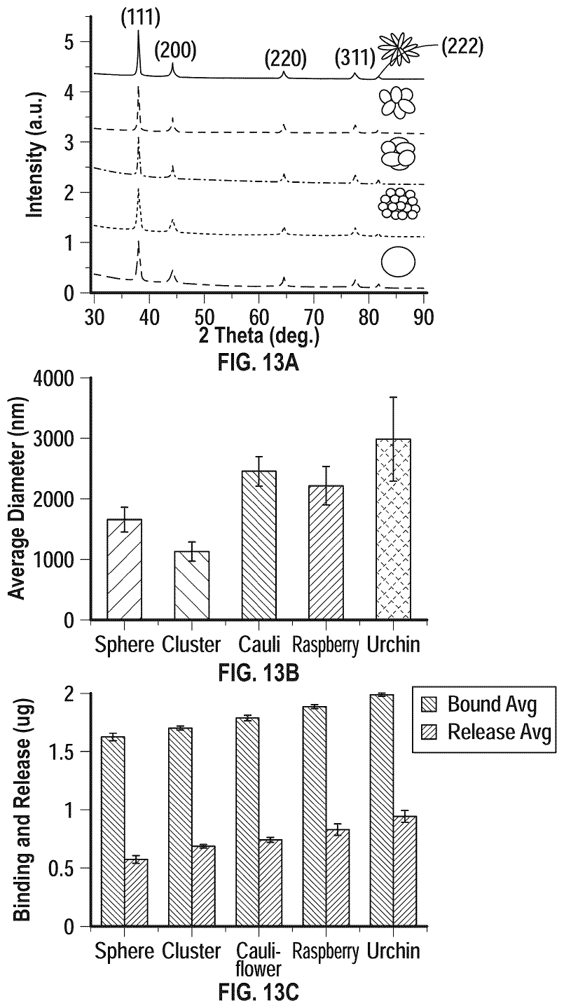

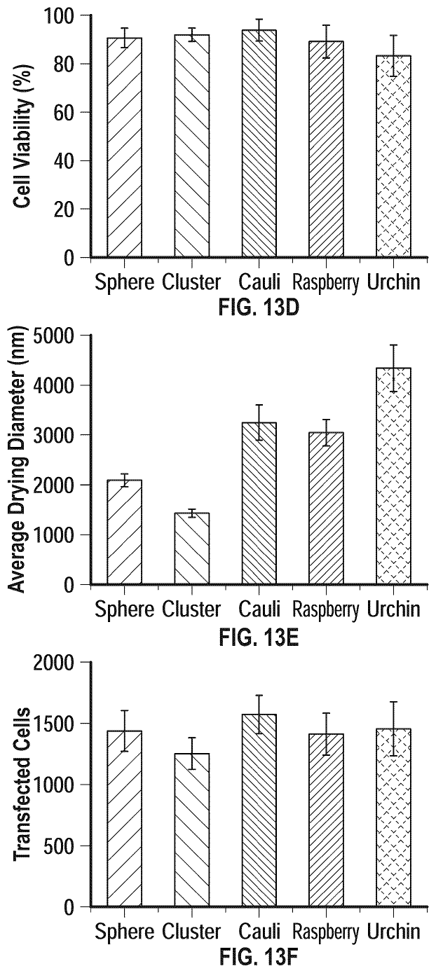

FIGS. 13A-F show characterization of different synthesized gold morphologies. FIG. 13C shows binding and release efficiency increases with more anisotropic structures (more surface area). FIG. 13F shows cauliflower morphology particles lead to the best performance, transfecting approximately ˜1575 cells on average. Spherical morphology was the second best at around ˜1450 cells.

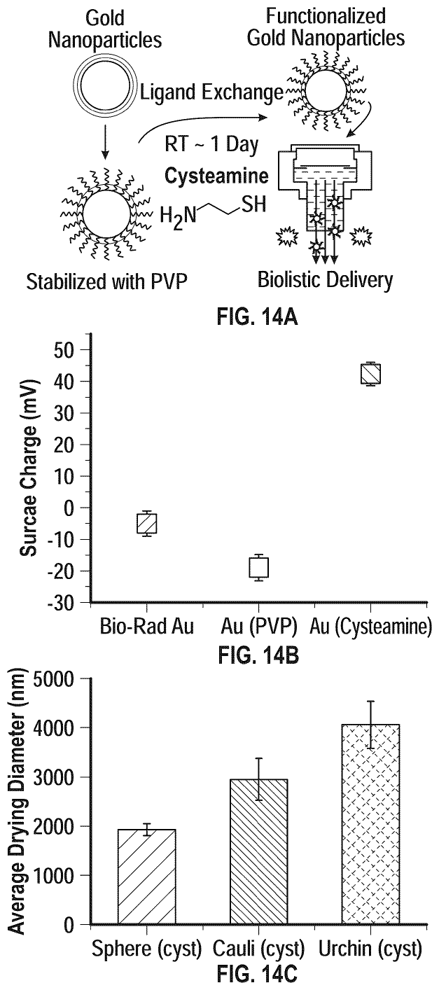

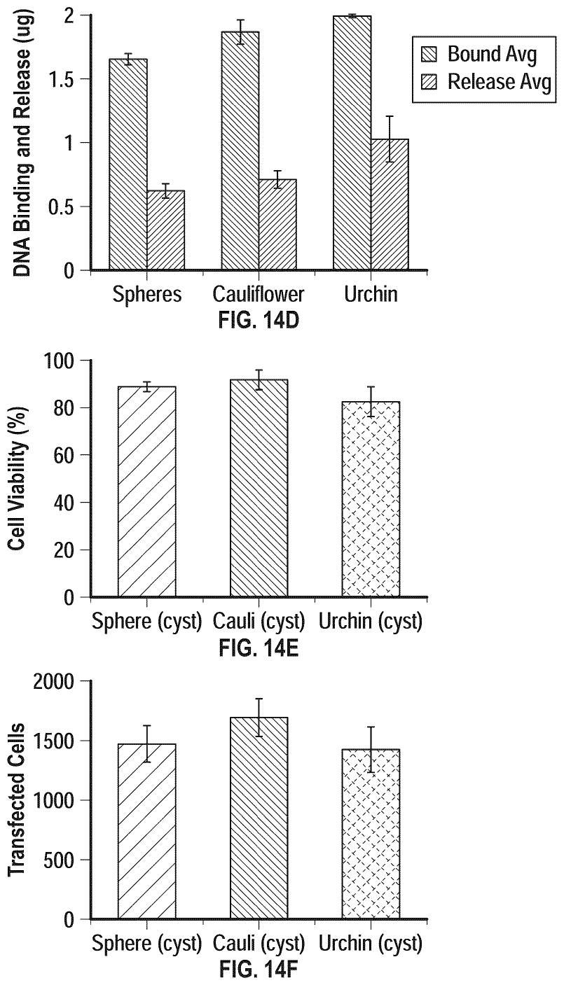

FIGS. 14A-14F show functionalization of synthesis spherical, cauliflower, and urchin morphologies. FIG. 14A shows ligand exchange with PVP to replace with cysteamine allows direct binding of DNA to the gold surface. An external delivery agent is no longer needed. FIG. 14B evidences surface charge shows functionalization works, wherein the cysteamine particles are very positively charged. FIG. 14D evidences rougher morphologies still show better binding and release. The functionalized particles bind and release slightly better than the plain particles with an external delivery agent. FIG. 14F show once again, functionalized cauliflower gold particles showed the best performance with approximately ˜1750 cells transfected on average. Functionalized spherical particles were the second best with around ˜1500 cells on average.

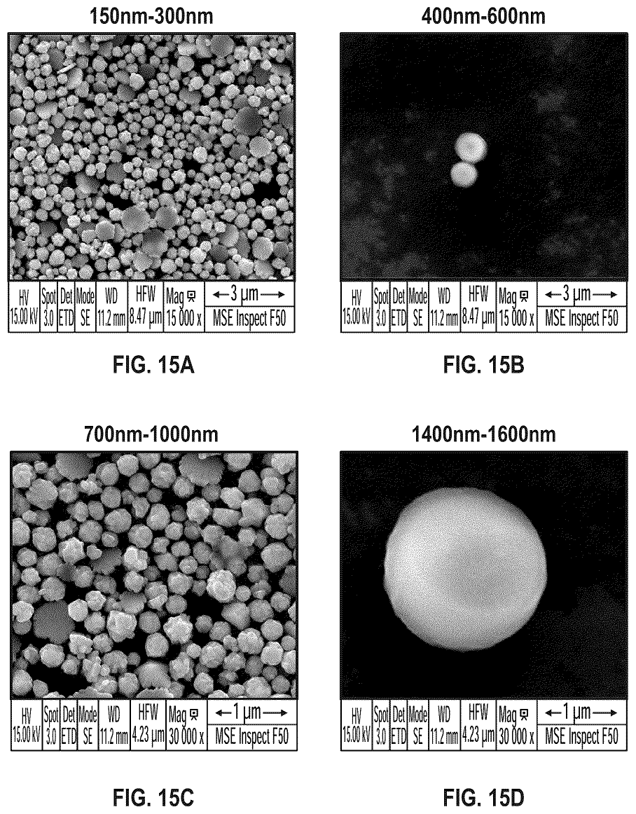

FIGS. 15A-15D show that substituting the gold precursor with another metallic precursors can still allow for a successful reaction. The synthesis method works across metals. For example, either a silver precursor (e.g., the silver nitrate that was used to form the silver particles shown in FIG. 15A and FIG. 15C) or a copper precursor (e.g., the copper(II) nitrate trihydrate that was used to form the copper particles of FIG. 15B and FIG. 15D) still allow for a successful bottom-up synthesis reaction. Such other metallic precursors include but are not limited to the use of a platinum precursor. It should be noted that these other metal particles synthesized (anything but gold) do not necessarily need be used for biolistic delivery, and likely have other commercial uses.

Synthesis of other transition metals with precursors that are water soluble are also possible, according to aspects of the present disclosure.

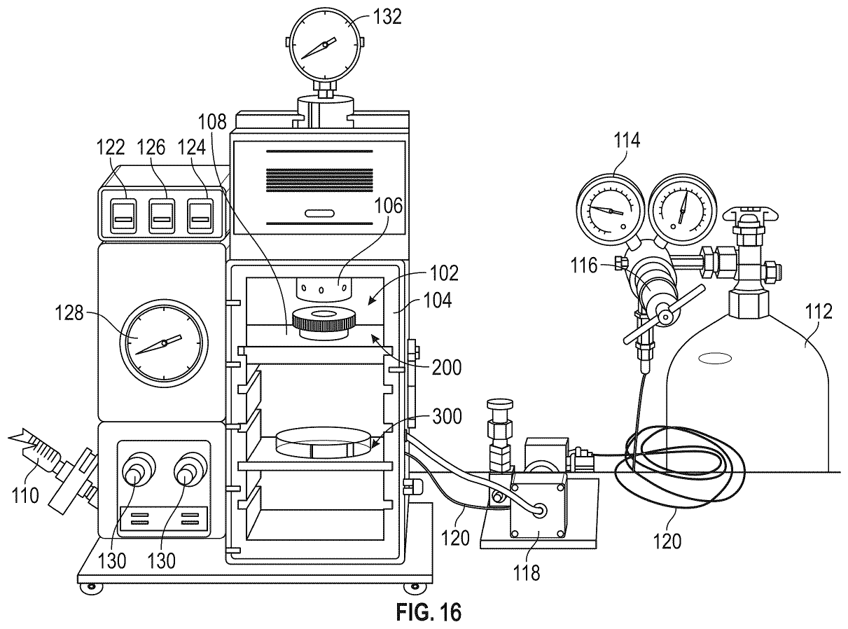

For example, the particle delivery system 100, the PDS-1000/He instrument, which is manufactured and sold by Bio-Rad Laboratories (Hercules, CA; hereinafter “Bio-Rad”), is shown in FIG. 16. The system 100 uses pressurized helium to accelerate subcellular sized microprojectiles coated with DNA or other biological material over a range of velocities necessary to optimally transform many different cell types. A microcarrier launch assembly 200 is placed within a bombardment chamber 102. Access to the bombardment chamber 102 is permitted through a bombardment chamber door 104. The bombardment chamber door 104, with a brace, controls supply of line electrical to the instrument. The bombardment chamber 102 also includes a rupture disk retaining cap 106, a launch assembly shelf 108, and a target plate shelf 300.

The system 100 also includes components for the attachment and delivery of high-pressure helium to the main unit. For example, the system 100 includes vacuum tubing 110 (e.g., reinforced polyvinyl chloride, PVC, tubing) for attachment to a vacuum source. A tank 112 stores pressurized helium. Delivery of helium is controlled by a helium regulator 114, a helium pressure regulator 116, a solenoid valve 118, and connective polyether ether ketone (“PEEK”) tubing 120.

With respect to the controls of the system 100, an on/off power switch 122 controls supply of line electrical power to the instrument. A fire switch 124 controls flow of helium into the gas acceleration tube by activating the solenoid valve 118. The fire switch 124 is illuminated red when enabled. When the safety interlock is satisfied that at least 5″ Hg vacuum is present in the chamber, the fire switch 124 must be held on continuously until rupture disk bursts and then released to stop the flow of helium. If the fire switch 124 is released before the disk ruptures, the helium is vented via a safety vent in the external 3-way metering (solenoid) valve 118. A vacuum/vent/hold switch assembly 126 controls application of vacuum to the bombardment chamber 102. Vacuum is applied from the line source. The vent releases vacuum using filtered air. The hold switch maintains vacuum by isolating chamber. Bombardments are performed with the hold switch in the ‘hold’ position. The vacuum gauge 128 indicates a level of vacuum in the bombardment chamber 102, in inches of mercury where zero equals ambient atmospheric pressure. The vacuum/vent rate control valves 130 regulate rate of application and relief of vacuum in bombardment chamber. Clockwise rotation closes valves. A helium pressure gauge 132 indicates helium pressure (in psi) in the gas acceleration tube.

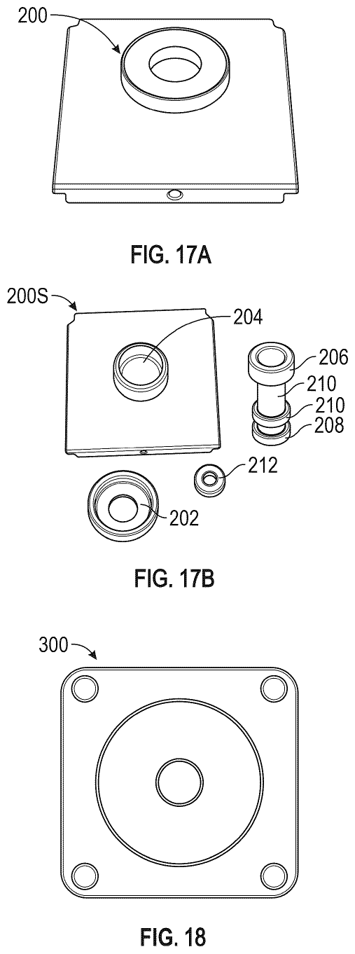

The microcarrier launch assembly 200 is shown in greater detail in FIGS. 17A-17B. The microcarrier launch assembly 200 is shipped fully assembled, as shown in FIG. 17A. The microcarrier launch assembly 200 comprises the launch assembly shelf 108 with a recessed set screw, a macrocarrier cover lid 202, an adjustable nest 204, a fixed nest 206 with a retaining spring, a stopping screen support ring 208, two spacer rings 210, and five macrocarrier holders 212. The macrocarrier holders 212 are for use within the microcarrier launch assembly 200 after the macrocarrier is inserted using a macrocarrier insertion tool.

The microcarrier launch assembly 200 comprises the launch assembly shelf 300 shown in FIG. 18. The target shelf 300 holds the biological target in a petri plate in the path of the accelerated DNA/microcarrier preparation. Particle flight distance is determined by positioning the shelf at one of four levels using slots in the chamber walls.

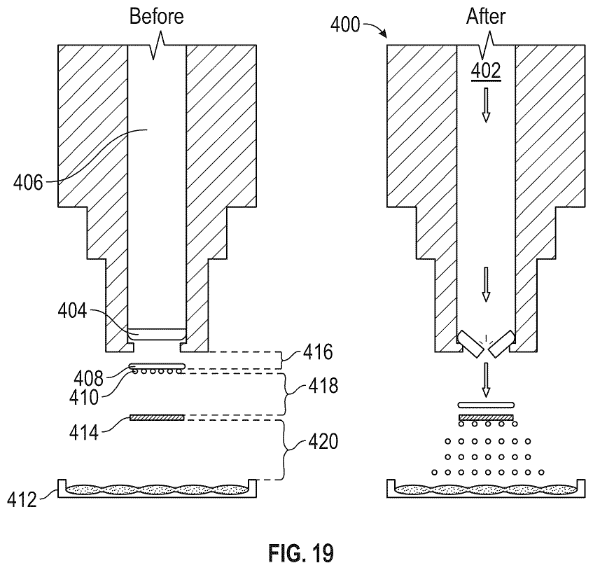

More particularly, the particle delivery system 100 shown in FIG. 16 operates using the ballistic process 400 shown in the before and after images shown of FIG. 19A and FIG. 19B. The ballistic process 400 utilizes high-pressure helium 402, released by a rupture disk 404, and partial vacuum (e.g., gas acceleration tube 406) to propel a macrocarrier sheet 408 loaded with millions of microscopic gold microcarriers 410 toward target cells 412 at a high velocity.

The rupture disk retaining cap 106 seals the rupture disk 404 against the chamber end of the gas acceleration tube 406. The rupture disk retaining cap 106 is tightened securely. The microcarrier launch assembly 200 holds the DNA/microcarrier preparation on the macrocarrier sheet 108 over the sheet over the stopping screen in the path of the helium shock wave. When the solenoid valve 118 is activated by the fire switch 124, the needle in the helium pressure gauge 132 (an oil-filled gauge) rotates clockwise until the rupture disk 404 bursts.

The microcarriers 410 are coated with DNA or other biological material for transformation. The macrocarrier 408 is halted after a short distance by a stopping screen 414. The DNA-coated microcarriers 410 continue traveling toward the target cells 412 to penetrate and transform the target cells 412.

The launch velocity of microcarriers 410 for each bombardment depends on the helium pressure (rupture disk 404 selection), the amount of vacuum in the bombardment chamber, the distance 416 from the rupture disk 404 to the macrocarrier sheet 408, the macrocarrier travel distance 418 to the stopping screen 414, and the distance 420 between the stopping screen 414 and the target cells 412.

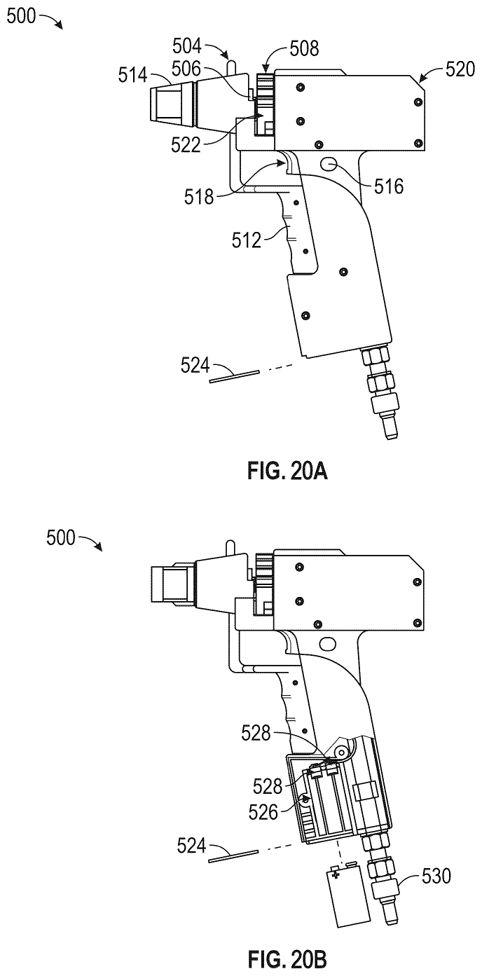

FIGS. 20A-20B show an example of a portable, helium driven gene gun 500, known as the Helios Gene Gun. The Helios Gene Gun is also manufactured and sold by Bio-Rad. The Helios Gene Gun contrasts the PDS-1000/He instrument because the overall size of the target to be transformed is limited by the size of the chamber and the target tissue is subjected to a vacuum during bombardment. The Helios Gene Gun requires no vacuum and any target accessible to the barrel can be transformed. Consequently, the Helios Gene Gun may be used in a much wider variety of gene transfer applications and provides a tool for both in vitro and in vivo transformations in the research lab. Essentially, any type of cells which can be made accessible to its nozzle may be transformed.

The helium driven gene gun 500 comprises components for preparing DNA-coated microcarriers, coating the DNA-microcarrier suspension onto the inner surface of the tubing, cutting the tubing into cartridges 502 which are used in the helium driven gene gun 500, and finally propel the microcarriers and their associated DNA into cells.

A cylinder lock 504 controls movement of the barrel pin 506. The cylinder lock 504 is spring-loaded. The natural position of the cylinder lock 504 is in the backward (locked) position so that the barrel pin 506 is inserted into the hole in the cartridge holder 508. This keeps the cartridge holder 506 in a proper position for firing. Moving the cylinder lock 504 forward disengages the barrel pin 506 from the cartridge holder 508 to permit removing the cartridge holder from the helium drive gene gun 500. Moving the cylinder lock forward and to the right latches the cylinder lock 506 to permit removal of the cartridge holder 508. However, to prevent damage to the O-rings 510, the cartridge holder 508 is only removed after compressing the cylinder advance lever 512.

The cylinder advance lever 512 is a multi-functional lever that is spring-activated by pulling the cylinder advance lever 512 backwards. When inserting or removing a cartridge holder 508, releasing the cylinder advance lever 512 moves the barrel liner 514 backward, bringing the O-ring 510 on the back of the liner 514 in contact with the cartridge holder 508. After discharging macrocarriers from one cartridge 502, pulling the cylinder advance lever 512 moves the barrel forward to increase the space for inserting the cartridge holder behind the barrel liner 514. This ratchets the cartridge holder 508, bringing the next cartridge 502 into firing position.

A safety interlock switch 516 is held down to permit the trigger button 518 to be operational. Once the interlock switch 516 is depressed, the trigger button 518 is functional for approximately thirty seconds. The LED display 520 flashes quickly during this time. If the trigger button 518 is not pressed within the allotted time, the safety interlock switch 516 must be released and pressed again to re-activate the trigger button 518.

The trigger button 518 controls the flow of helium gas through the gene gun 500. The trigger button 518 acts as a switch and momentarily activates a solenoid to open a main valve, permitting helium to enter the cartridge 502 and barrel. The trigger button 518 only activates for a limited time after the safety interlock switch 516 is depressed.

A metal bar 522 serves as the mechanism that ratchets the cartridge holder 508 from one position to the next when the cylinder advance lever 512 is pressed. When the cylinder advance lever 512 is moved outward prior to inserting a cartridge holder 508, additional room is provided for maneuvering the cartridge holder 508.

The electrical system of the helium driven gene gun 500 is powered by a battery, which is accessed by way of a battery access cover 524. Under normal use, a nine volt (9V) battery should provide sufficient energy for 1000 shots. As shown in FIG. 20B, the battery compartment 526 is in the base of the handle near the attachment fitting 530 for the helium hose. The battery compartment 526 is protected by the battery access cover 524 that slides forward. The battery is inserted with the positive terminal (the smaller of the two terminals 528) facing forward.

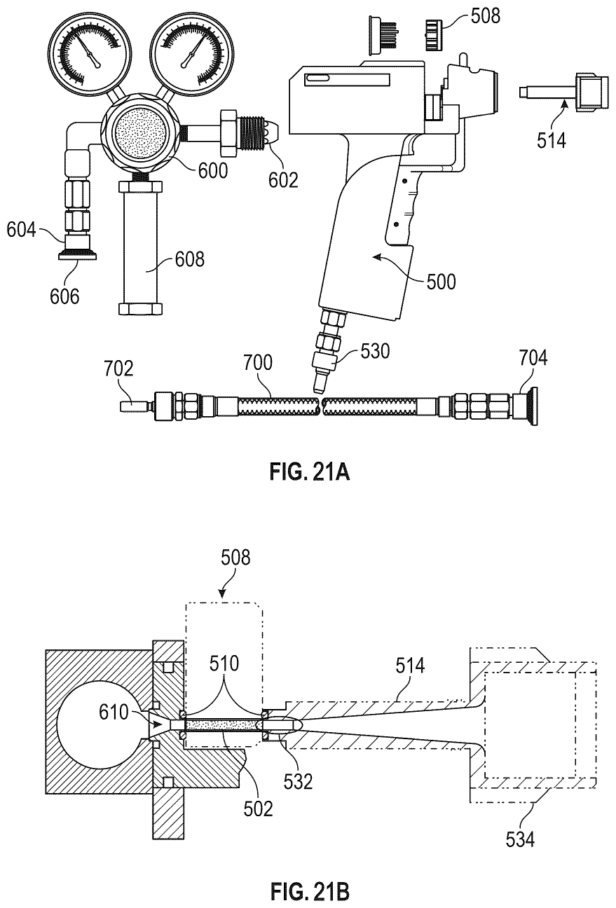

FIG. 21A shows how the helium driven gene gun 500 interacts with other major components used for sample delivery, such as the helium regulator 600 (which includes a connection 602 to the helium tank and a sleeve 604) and the helium hose 700. The helium drive gene gun 500 can be prepared for firing, discharging the device, loading cartridges into the cartridge holder 504, and delivering DNA to target cells.

More particularly, the attachment fitting 530 of the helium gun 500 can be inserted into an opening on the body of the female quick-connect fitting 704 on the helium hose 700 and pushed until it clicks or otherwise locks. The helium hose 700 can then be locked into the helium regulator 600 by inserting a stem 702 into the female quick connect fitting 606 on the helium regulator 502 until it clicks or otherwise locks. As a result of the two connections, the helium driven gene gun 500 is indirectly locked into the helium regulator 600. The pressure relief valve 608 can be turned counter-clockwise to depressurize the system in the even the helium regulator 600 has been pressurized prior to the intended time and/or the stem 702 and body 606 will not lock.

In use, and prior to transfection, the plasmid DNA is attached to the gold particles. This is accomplished by precipitation of the DNA from solution in the presence of gold microcarriers and the polycarbon spermidine by the addition of CaCl₂. The particles are then washed extensively with ethanol to remove the water and resuspended in ethanol. The DNA/microcarrier solution is coated onto the inner wall of Gold-Coat tubing and dried. The tubing is cut into 0.5″ length cartridges 502. These cartridges 502, when inserted into the cartridge holder 508 of the helium driven gene gun 500 are the source of the DNA which enters the target cells by the helium discharge.

The helium driven gene gun 500 employs a high velocity stream of helium 610 to accelerate gold particles coated with plasmids or RNA to velocities sufficient to penetrate and transform cells, both in vitro and in vivo, as shown in FIG. 21B. The discharge is initiated by pressing the trigger button 518 which activates the main valve, causing helium to travel down the bore of the particle delivery device (gene gun 500). When the helium enters one of the bores of the cylinder containing the cartridge 502, the gold particles on the inside of the tubing are pulled from the surface, become entrained in the helium stream 610, and begin to pick up speed. Immediately past the acceleration channel 532, the barrel begins to open as a cone. The slope of the cone causes the gas to be pulled outward, expanding the high-pressure stream 610 into a less destructive low velocity pulse, while the gold particles maintain a high velocity. The expansion also helps spread the microcarriers from their original diameter to an area approximately four times greater in diameter at the target site.

Helium gas is pulsed through the cartridge loaded with DNA-coated-microcarriers. This pulse sweeps the microcarriers from the inside wall of the cartridge. As the microcarriers enter the barrel liner 514 they pick up speed in the acceleration channel 532 then spread out as they travel down the barrel; the increased cross-sectional area of the barrel from the acceleration chamber 532 to the spacer 534 also moderates the helium shock wave so it is less intense when it reaches the target cells. The O-rings 510 on each side of the cartridge holder 508 direct the flow of helium 610 through the cartridge 502 and the acceleration channel 532. The spacer 534 maintains target distance and permits venting of the helium gas away from the target.

The present inventors have enhanced and improved such biolistic delivery systems, as shown and described in U.S. Pre-grant Pub. No. 2024/0010964 A1, which is hereby incorporated by reference in its entirety herein.

From the foregoing, it can be seen that the present disclosure accomplishes at least all of the stated objectives.

### GLOSSARY

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A bottom-up synthesis reaction to form gold particles having a diameter between three hundred nanometers and six microns (300 nm-6 μm), comprising:
a capping agent;
an amine containing reducing agent; and
a gold precursor;
wherein a temperature of the bottom-up synthesis reaction is between zero degrees Celsius (0° C.) and one hundred degrees Celsius (100° C.).

2. The bottom-up synthesis reaction of claim 1, wherein the amine containing reducing agent comprises a benzene ring.

3. The bottom-up synthesis reaction of claim 1, wherein the gold precursor is selected from the group consisting of: gold (III) chloride hydrate (HAuCl4), Chloroauric Acid (HAuCl₄), Gold(I) Chloride (AuCl), Gold(I) Thiosulfate (Au(S₂O₃)₂³⁻), Gold(III) Nitrate (Au(NO₃)₃), Gold(III) Acetate (Au(C₂H₃O₂)₃), Gold(I) Thioglucose (Au(TG)), Tetrafluoroborate (AuBF₄), and Gold(III) Sulfate (Au₂(SO₄)₃).

4. The bottom-up synthesis reaction of claim 1, wherein the capping agent is selected from the group consisting of: polyvinylpyrrolidone (PVP), hydroxyethyl cellulose (HEC), polyvinyl alcohol (PVA), polyethylene glycol (PEG).

5. The bottom-up synthesis reaction of claim 1, wherein the amine containing reducing agent is selected from the group consisting of: 4-aminophenol and o-phenylenediamine.

6. The bottom-up synthesis reaction of claim 1, wherein the temperature of the reaction is a room temperature between 20° C. and 24° C.

7. The bottom-up synthesis reaction of claim 1, wherein the capping agent comprises cysteamine and the synthesis reaction is free from a binding agent and salt for complexing with DNA.

8. The bottom-up synthesis reaction of claim 1, wherein the yield of the bottom-up synthesis reaction is approximately one hundred percent.

9. A method comprising:
using a synthesis reaction to form gold particles having a diameter between three hundred nanometers and six microns (300 nm-6 μm), wherein a temperature of the reaction is between zero degrees Celsius (0° C.) and one hundred degrees Celsius (100° C.); and
surface functionalizing the gold particles with a binding agent.

10. The method of claim 9, wherein the binding agent comprises highly positively charged molecules.

11. The method of claim 9, further comprising capping the gold particles with cysteamine.

12. The method of claim 9, wherein the gold particles are monodisperse gold particles.

13. The method of claim 9, further comprising stirring the reaction at a rate between three hundred and two thousand revolutions per minute (300 rpm-2000 rpm).

14. The method of claim 9, wherein the gold particles are synthesized in less than thirty minutes.

15. The method of claim 9, further comprising controlling a size and a morphology of the gold particles through selecting a concentration or a volume of an amine containing reducing agent and a capping agent used in the synthesis reaction.

16. A biolistic delivery system for delivery the gold particles synthesized using the method of claim 9.

17. The biolistic delivery system of claim 16, wherein the gold particles are used for causing a transient transformation, a stable transformation, or gene editing with DNA, RNA, or a protein.

18. A bottom-up synthesis reaction to form particles having a diameter between three hundred nanometers and six microns (300 nm-6 μm), comprising:
a capping agent;
an amine containing reducing agent; and
a metallic precursor or a water-soluble precursor;
wherein a temperature of the bottom-up synthesis reaction is between zero degrees Celsius (0° C.) and one hundred degrees Celsius (100° C.).

19. A transition metal formed with a bottom-up synthesis reaction of claim 18.
